(19) United States
(12) Patent Application Publication
(10) Pub. No.: US 2005/0033052 A1
(43) Pub. Date: Feb. 10, 2005

(54) Novel compounds

(76) Inventors: Colin Andrew Leach, King of Prussia, PA (US); Ivan Leo Pinto, Stevenage (GB); Stephen Allan Smith, Stevenage (GB); Steven James Stanway, Harlow (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/495,025

(22) PCT Filed: Nov. 8, 2002
(86) PCT No.: PCT/EP02/12506

(30) Foreign Application Priority Data
Nov. 10, 2001 (GB) 0127140.2

(51) Int. Cl. C07D 213/69; C07D 239/02

(52) U.S. Cl. 544/309; 546/296

(57) ABSTRACT

Pyrimidone and pyridone compounds of the formula: 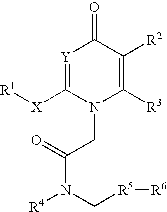
are inhibitors of the enzyme Lp-PLA₂ and are of use in therapy, in particular for treating atherosclerosis.

The present invention relates to certain novel pyrimidone and pyridone compounds, processes for their preparation, intermediates useful in their preparation, pharmaceutical compositions containing them and their use in therapy, in particular in the treatment of atherosclerosis.

WO 95/00649 (SmithKline Beecham plc) describes the phospholipase A₂ enzyme Lipoprotein Associated Phospholipase A₂ (Lp-PLA₂), the sequence, isolation and purification thereof, isolated nucleic acids encoding the enzyme, and recombinant host cells transformed with DNA encoding the enzyme. Suggested therapeutic uses for inhibitors of the enzyme included atherosclerosis, diabetes, rheumatoid arthritis, stroke, myocardial infarction, reperfusion injury and acute and chronic inflammation. A subsequent publication from the same group further describes this enzyme (Tew D et al, Arterioscler Thromb Vas Biol 1996:16;591-9) wherein it is referred to as LDL-PLA₂. A later patent application (WO 95/09921, Icos Corporation) and a related publication in Nature (Tjoelker et al, vol 374, 6 April 1995, 549) describe the enzyme PAF-AH which has essentially the same sequence as Lp-PLA₂ and suggest that it may have potential as a therapeutic protein for regulating pathological inflammatory events.

It has been shown that Lp-PLA₂ is responsible for the conversion of phosphatidylcholine to lysophosphatidylcholine, during the conversion of low density lipoprotein (LDL) to its oxidised form. The enzyme is known to hydrolyse the sn-2 ester of the oxidised phosphatidylcholine to give lysophosphatidylcholine and an oxidatively modified fatty acid. Both products of Lp-PLA₂ action are biologically active with lysophosphatidylcholine in particular having several pro-atherogenic activities ascribed to it, including monocyte chemotaxis and induction of endothelial dysfunction, both of which facilitate monocyte-derived macrophage accumulation within the artery wall. Inhibition of the Lp-PLA₂ enzyme would therefore be expected to stop the build up of these macrophage enriched lesions (by inhibition of the formation of lysophosphatidylcholine and oxidised free fatty acids) and so be useful in the treatment of atherosclerosis.

The increased lysophosphatidylcholine content of oxidatively modified LDL is also thought to be responsible for the endothelial dysfunction observed in patients with atherosclerosis. Inhibitors of Lp-PLA₂ could therefore prove beneficial in the treatment of this phenomenon. An Lp-PLA₂ inhibitor could also find utility in other disease states that exhibit endothelial dysfunction including diabetes, hypertension, angina pectoris and after ischaemia and reperfusion.

In addition, Lp-PLA₂ inhibitors may also have a general application in any disorder that involves activated monocytes, macrophages or lymphocytes, as all of these cell types express Lp-PLA₂. Examples of such disorders include psoriasis.

Furthermore, Lp-PLA₂ inhibitors may also have a general application in any disorder that involves lipid oxidation in conjunction with Lp-PLA₂ activity to produce the two injurious products, lysophosphatidylcholine and oxidatively modified fatty acids. Such conditions include the aforementioned conditions atherosclerosis, diabetes, rheumatoid arthritis, stroke, myocardial infarction, ischaemia, reperfusion injury and acute and chronic inflammation.

Patent applications WO 96/12963, WO 96/13484, WO96/19451, WO 97/02242, WO97/217675, WO 97/217676, WO 96/41098, and WO 97/41099 (SmithKline Beecham plc) disclose inter alia various series of 4-thionyl/sulfinyl/sulfonyl azetidinone compounds which are inhibitors of the enzyme Lp-PLA₂. These are irreversible, acylating inhibitors (Tew et al, Biochemistry, 37, 10087, 1998).

A further class of compounds has now been identified which are non-acylating inhibitors of the enzyme Lp-PLA₂. Thus, WO 99/24420, WO 00/10980, WO 00/66566, WO 00/66567 and WO 00/68208 (SmithKline Beecham plc) disclose a class of pyrimidone compounds which are exemplified by an optionally substituted 2-benzylthio or 2-benzyloxy substituent. We have now found that the pyrimidone ring, optionally replaced by a pyridone ring, may be fused to a heterocyclyl ring to give compounds having good activity as inhibitors of the enzyme Lp-PLA₂.

Accordingly, the present invention provides a compound of formula (I): 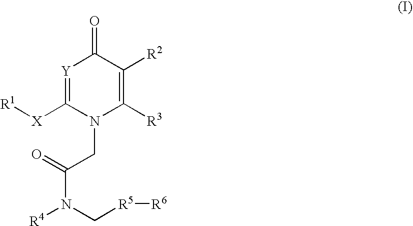
in which:
R¹ is an aryl group, optionally substituted by 1, 2, 3 or 4 substituents which may be the same or different selected from C₍₁₋₆₎alkyl, C₍₁₋₆₎alkoxy, C₍₁₋₆₎alkylthio, hydroxy, halogen, CN, and mono to perfluoro-C₍₁₋₄₎alkyl;
R² and R³ together with the ring carbon atoms to which they are attached form a fused 5- or 6-membered heterocyclyl ring containing 1 or 2 heteroatoms which may be the same or different selected from N, O and S, optionally substituted by 1, 2 or 3 substituents which may be the same or different selected from oxo, hydroxy, halogen, OR⁷, COR⁷, COOR⁷, CONR⁹R¹⁰, SR⁷, NR⁷COR⁸, SO₂NR⁹R¹⁰, NR⁷SO₂R⁸, and C₍₁₋₆₎alkyl optionally substituted by 1, 2 or 3 substituents selected from hydroxy, halogen, OR⁷, COR⁷, carboxy, COOR⁷, CONR⁹R¹⁰ and NR⁹R¹⁰;
R⁴ is hydrogen, C₍₁₋₆₎alkyl which may be unsubstituted or substituted by 1, 2 or 3 substituents selected from hydroxy, halogen, OR⁷, COR⁷, carboxy, COOR⁷, CONR⁹R¹⁰, NR⁹R¹⁰, NR⁷COR⁸, mono- or di-(hydroxyC₍₁₋₆₎alky)amino and N-hydroxyC₍₁₋₆₎alkyl-N—C₍₁₋₆₎alkylamino; or
R⁴ is Het-C₍₀₋₄₎alkyl in which Het is a 5- to 7-membered heterocyclyl ring comprising N and optionally O or S, and in which N may be substituted by COR⁷, COOR⁷, CONR⁹R¹⁰, or C₍₁₋₆₎allyl optionally substituted by 1, 2 or 3 substituents selected from hydroxy, halogen, OR⁷, COR⁷, carboxy, COOR⁷, CONR⁹R¹⁰ and NR⁹R¹⁰, for instance, piperidin-4-yl, pyrrolidin-3-yl;
R⁵ is an aryl or a heteroaryl ring optionally substituted by 1, 2, 3 or 4 substituents which may be the same or different selected from C₍₁₋₆₎alkyl, C₍₁₋₆₎alkoxy, C₍₁₋₆₎alkylthio, arylC₍₁₋₆₎alkoxy, hydroxy, halogen, CN, COR⁷, carboxy, COOR⁷, NR⁷COR⁸, CONR⁹R¹⁰, SO₂NR⁹R¹⁰, NR⁷SO₂R⁸, NR⁹R¹⁰, mono to perfluoro-C₍₁₋₄₎allyl and mono to perfluoro-C₍₁₋₄₎alkoxy;
R⁶ is an aryl or a heteroaryl ring which is further optionally substituted by 1, 2, 3 or 4 substituents which may be the same or different selected from C₍₁₋₁₈₎alkyl, C₍₁₋₁₈₎alkoxy, C₍₁₋₆₎alkylthio, C₍₁₋₆₎alkylsulfonyl, arylC₍₁₋₆₎alkoxy, hydroxy, halogen, CN, COR⁷, carboxy, COOR⁷, CONR⁹R¹⁰, NR⁷COR⁸, SO₂NR⁹R¹⁰, NR⁷SO₂R⁸, NR⁹R¹⁰, mono to perfluoro-C₍₁₋₄₎alkyl and mono to perfluoro-C₍₁₋₄₎alkoxy, or C₍₅₋₁₀₎alkyl;
R⁷ and R⁸ are independently hydrogen or C₍₁₋₁₂₎alkyl, for instance C₍₁₋₄₎allyl (e.g. methyl or ethyl);
R⁹ and R¹⁰ which may be the same or different is each selected from hydrogen, or C₍₁₋₁₂₎alkyl, or R⁹ and R¹⁰ together with the nitrogen to which they are attached form a 5- to 7 membered ring optionally containing one or more further heteroatoms selected from oxygen, nitrogen and sulphur, and optionally substituted by one or two substituents selected from hydroxy, oxo, C₍₁₋₄₎alkyl, C₍₁₋₄₎alkylcarboxy, aryl, e.g. phenyl, or aralkyl, e.g benzyl, for instance morpholine or piperazine;
X is C₍₂₋₄₎alkylene, optionally substituted by 1, 2 or 3 substituents selected from methyl and ethyl, CH═CH or (CH₂)ₙS where n is 1, 2 or 3; and
Y is CH or N.

In one aspect the aryl group of R¹ may be phenyl or naphthyl. Preferably, R¹ is phenyl optionally substituted by halogen, C₍₁₋₆₎alkyl, trifluoromethyl, C₍₁₋₆₎alkoxy, preferably, from 1 to 3 fluoro, more preferably, 2,3-difluoro.

In another aspect R² and R³ together with the ring carbon atoms to which they are attached may form a fused 5- or 6-membered heterocyclyl ring containing a sulphur atom, a nitrogen atom or an oxygen atom, particularly a fused 5-membered heterocyclyl ring.

In another aspect R⁴ may be hydrogen, methyl, 2-(diethylamino)ethyl, 2-(piperidin-1-yl)ethyl, 2-(pyrrolidin-1-yl)ethyl, 1-methyl-piperidinyl, 1-ethyl-piperidin-4-yl, 1-ethyl-pyrrolidin-2-ylmethyl or 1-(2-methoxyethyl)piperidin-4-yl. Preferably R⁴ is 2-(diethylamino)ethyl, 1-ethyl-piperidin-4-yl or 1-(2-methoxyethyl)piperidinyl.

In another aspect R⁵ may be phenyl or pyridyl. Preferably, R⁵ is phenyl.

In another aspect R⁶ may be phenyl optionally substituted by halogen, or trifluoromethyl, preferably at the 4-position, or ethyl. Preferably, R⁶ is phenyl substituted by trifluoromethyl at the 4-position.

Preferably, R⁵ and R⁶ together form a 4-(phenyl)phenyl or a 2-(phenyl)pyridinyl substituent in which the remote phenyl ring may be optionally substituted by halogen or trifluoromethyl, preferably at the 4-position.

Preferably X is C₍₂₋₄₎alkylene, more preferably C₍₂₋₃₎alkylene, most preferably, (CH₂)₂.

Preferably Y is N.

It will be appreciated that compounds of the present invention may comprise one or more chiral centres so that stereoisomers may be formed. The present invention encompasses all stereoisomers of the compounds of formula (I) including geometric isomers and optical isomers (eg. diastereoisomers and enantiomers) whether as individual stereoisomers isolated such as to be substantially free of the other stereoisomers (ie. pure) or as mixtures thereof including racemic modifications. An individual stereoisomer isolated such as to be substantially free of other stereoisomer (ie. pure) will preferably be isolated such that less than 10% preferably less than 1% especially less than 0.1% of the other stereoisomers is present.

Certain compounds of formula (I) may exist in one of several tautomeric forms. It will be understood that the present invention encompasses all tautomers of the compounds of formula (I) whether as individual tautomers or as mixtures thereof.

It will be appreciated that in some instances, compounds of the present invention may include a basic function such as an amino group as a substituent. Such basic functions may be used to form acid addition salts, in particular pharmaceutically acceptable salts. Pharmaceutically acceptable salts include those described by Berge, Bighley, and Monkhouse, J. Pharm. Sci., 1977, 66, 1-19. Such salts may be formed from inorganic and organic acids. Representative examples thereof include maleic, fumaric, benzoic, ascorbic, pamoic, succinic, bismethylenesalicylic, methanesulfonic, ethanedisulfonic, acetic, propionic, tartaric, salicylic, citric, gluconic, aspartic, stearic, palmitic, itaconic, glycolic, p-aminobenzoic, glutamic, taurocholic acid, benzenesulfonic, p-toluenesulfonic, hydrochloric, hydrobromic, sulfuric, cyclohexylsulfamic, phosphoric and nitric acids.

It will be appreciated that in some instances, compounds of the present invention may include a carboxy group as a substituent. Such carboxy groups may be used to form salts, in particular pharmaceutically acceptable salts. Pharmaceutically acceptable salts include those described by Berge, Bighley, and Monkhouse, J. Pharm. Sci., 1977, 66, 1-19. Preferred salts include alkali metal salts such as the sodium and potassium salts.

When used herein, the term “alkyl” and similar terms such as “alkoxy” includes all straight chain and branched isomers. Representative examples thereof include methyl, ethyl, n-propyl, iso-propyl, n-butyl, sec-butyl, iso-butyl, t-butyl, n-pentyl and n-hexyl.

When used herein, the term “aryl” refers to, unless otherwise defined, a mono- or bicyclic aromatic ring system containing up to 10 carbon atoms in the ring system, for instance phenyl or naphthyl.

When used herein, the term “heteroaryl” refers to a mono- or bicyclic heteroaromatic ring system comprising up to four, preferably 1 or 2, heteroatoms each selected from oxygen, nitrogen and sulphur. Each ring may have from 4 to 7, preferably 5 or 6, ring atoms. A bicyclic heteroaromatic ring system may include a carbocyclic ring.

When used herein, the terms “halogen” and “halo” include fluorine, chlorine, bromine and iodine and fluoro, chloro, bromo and iodo, respectively.

When used herein, the term “heterocyclyl” refers to a non-aromatic ring comprising one or two heteroatoms.

It is to be understood that the present invention covers all combinations of substituent groups referred to hereinabove.

A representative compound of formula (I) is N-(2-diethylaminoethyl)-2-(2-(2,3-difluorophenyl)ethyl)-4-oxo-5,7-dihydro-4H-thieno[3,4-d]pyrimidin-1-yl)-N-4-(4-trifluoromethylphenyl)benzyl)acetamide or a pharmaceutically acceptable salt thereof, in particular the bitartrate salt.

Since the compounds of the present invention, in particular compounds of formula (I), are intended for use in pharmaceutical compositions, it will be understood that they are each provided in substantially pure form, for example at least 50% pure, more suitably at least 75% pure and preferably at least 95% pure (% are on a wt/wt basis). Impure preparations of the compounds of formula (I) may be used for preparing the more pure forms used in the pharmaceutical compositions. Although the purity of intermediate compounds of the present invention is less critical, it will be readily understood that the substantially pure form is preferred as for the compounds of formula (I). Preferably, whenever possible, the compounds of the present invention are obtained in crystalline form.

When some of the compounds of this invention are allowed to crystallise or are re-crystallised from organic solvents, solvent of crystallisation may be present in the crystalline product. This invention includes within its scope such solvates. Similarly, some of the compounds of this invention may be crystallised or re-crystallised from solvents containing water. In such cases water of hydration may be formed. This invention includes within its scope stoichiometric hydrates as well as compounds containing variable amounts of water that may be produced by processes such as lyophilisation. In addition, different crystallisation conditions may lead to the formation of different polymorphic forms of crystalline products. This invention includes within its scope all polymorphic forms of the compounds of formula (I).

Compounds of the present invention are inhibitors of the enzyme lipoprotein associated phospholipase A₂ (Lp-PLA₂) and as such are expected to be of use in therapy, in particular in the treatment of atherosclerosis. In a further aspect therefore the present invention provides a compound of formula (I) for use in therapy.

The compounds of formula (I) are inhibitors of lysophosphatidylcholine production by Lp-PLA₂ and may therefore also have a general application in any disorder that involves endothelial dysfunction, for example atherosclerosis, diabetes, hypertension, angina pectoris and after ischaemia and reperfusion. In addition, compounds of formula (I) may have a general application in any disorder that involves lipid oxidation in conjunction with enzyme activity, for example in addition to conditions such as atherosclerosis and diabetes, other conditions such as rheumatoid arthritis, stroke, inflammatory conditions of the brain such as Alzheimer's Disease, myocardial infarction, reperfusion injury, sepsis, and acute and chronic inflammation.

Further applications include any disorder that involves activated monocytes, macrophages or lymphocytes, as all of these cell types express Lp-PLA₂. Examples of such disorders include psoriasis.

Accordingly, in a further aspect, the present invention provides for a method of treating a disease state associated with activity of the enzyme Lp-PLA₂ which method involves treating a patient in need thereof with a therapeutically effective amount of an inhibitor of the enzyme. The disease state may be associated with the increased involvement of monocytes, macrophages or lymphocytes; with the formation of lysophosphatidylcholine and oxidised free fatty acids; with lipid oxidation in conjunction with Lp PLA₂ activity; with ischemia and reperfusion; or with endothelial dysfunction.

Compounds of the present invention may also be of use in treating the above mentioned disease states in combination with an anti-hyperlipidaemic, anti-atherosclerotic, anti-diabetic, anti-anginal, anti-inflammatory, or anti-hypertension agent or an agent for lowering Lp(a). Examples of the above include cholesterol synthesis inhibitors such as statins, anti-oxidants such as probucol, insulin sensitisers, calcium channel antagonists, and anti-inflammatory drugs such as NSAIDs. Examples of agents for lowering Lp(a) include the aminophosphonates described in WO 97/02037, WO 98/28310, WO 98/28311 and WO 98/28312 (Symphar SA and SmithKline Beecham).

A preferred combination therapy will be the use of a compound of the present invention and a statin. The statins are a well known class of cholesterol lowering agents and include atorvastatin, simvarstatin, pravastatin, cerivastatin, fluvastatin, lovastatin and ZD 4522 (also referred to as S-4522, rosuvastatin, Astra Zeneca). The two agents may be administered at substantially the same time or at different times, according to the discretion of the physician.

A further preferred combination therapy will be the use of a compound of the present invention and an anti-diabetic agent or an insulin sensitiser, as coronary heart disease is a major cause of death for diabetics. Within this class, preferred compounds for use with a compound of the present invention include the PPARgamma activators, for instance GI262570 (GlaxoSmithKline) and the glitazone class of compounds such as rosiglitazone (Avandia, GlaxoSmithKline), troglitazone and pioglitazone.

In therapeutic use, the compounds of the present invention are usually administered in a standard pharmaceutical composition. The present invention therefore provides, in a further aspect, a pharmaceutical composition comprising a compound of formula (I) and a pharmaceutically acceptable carrier, optionally with one or more other therapeutic compounds such as a statin or an anti-diabetic.

Suitable pharmaceutical compositions include those which are adapted for oral or parenteral administration or as a suppository.

Compounds of formula (I) which are active when given orally can be formulated as liquids, for example syrups, suspensions or emulsions, tablets, capsules and lozenges. A liquid formulation will generally consist of a suspension or solution of the compound or pharmaceutically acceptable salt in a suitable liquid carrier(s) for example, ethanol, glycerine, non-aqueous solvent, for example polyethylene glycol, oils, or water with a suspending agent, preservative, flavouring or colouring agent. A composition in the form of a tablet can be prepared using any suitable pharmaceutical carrier(s) routinely used for preparing solid formulations. Examples of such carriers include magnesium stearate, starch, lactose, sucrose and cellulose. A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule. Typical parenteral compositions consist of a solution or suspension of the compound of formula (I) in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration. A typical suppository formulation comprises a compound of formula (I) which is active when administered in this way, with a binding and/or lubricating agent such as polymeric glycols, gelatins or cocoa butter or other low melting vegetable or synthetic waxes or fats.

Preferably the composition is in unit dose form such as a tablet or capsule. Each dosage unit for oral administration contains preferably from 1 to 500 mg (and for parenteral administration contains preferably from 0.1 to 25 mg) of a compound of the formula (I). The daily dosage regimen for an adult patient may be, for example, an oral dose of between 1 mg and 1000 mg, preferably between 1 mg and 500 mg, or an intravenous, subcutaneous, or intramuscular dose of between 0.1 mg and 100 mg, preferably between 0.1 mg and 25 mg, of the compound of the formula (I), the compound being administered 1 to 4 times per day. Suitably the compounds will be administered for a period of continuous therapy, for example for a week or more.

According to a first process (A), a compound of formula (I) may be prepared by reacting an acid compound of formula (II): 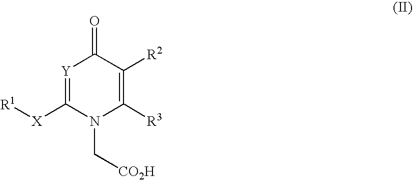
in which X, Y, R¹, R² and R³ are as hereinbefore defined,
with an amine compound of formula (III):
R⁶—R⁵—CH₂NHR⁴ (III)
in which R⁴, R⁵ and R⁶ are as hereinbefore defined; under amide forming conditions.

Suitable amide forming conditions are well known in the art and include treating the acid of formula (II) with the amine of formula (III) in the presence of a coupling agent such as 1-(3-dimethyl-aminopropyl)-3-ethylcarbodiimide (DEC).

A compound of formula (II) may be readily prepared from a corresponding unsubstituted compound of formula (IV): 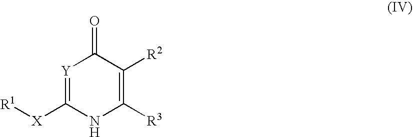
in which X, Y, R¹, R² and R³ are as hereinbefore defined,
by reaction with a compound of formula (V):
LCH₂CO₂R¹¹ (V)
in which L is a leaving group such as trifluoromethanesulphonate or halo, for example, chloro, bromo or iodo, and R¹¹ is C₍₁₋₆₎alkyl, for example ethyl or t-butyl, in the presence of a base such as a tertiary amine, for example di-isopropylethylamine; to form an intermediate ester (VI), 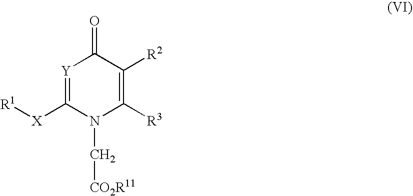
in which X, Y, R¹, R², R³ and R¹¹ are as hereinbefore defined,
and thereafter, removing R¹¹ by treating with a de-esterifying agent, for instance, for t-butyl, trifluoroacetic acid.

It will be appreciated that removal of R¹¹ may be carried out as a separate step, so that an acid of formula (II), or a salt thereof, for example the sodium salt, is isolated or, alternatively, that the acid of formula (II), or a salt thereof, is formed from the intermediate ester (VI), prior to reaction with an amine of formula (III).

Thus according to a further process B, a compound of formula (I) may be prepared by (a) treating a compound of formula (VI) with a de-esterifying agent to form a compound of formula (II) and (b) reacting said compound of formula (II) with an amine of formula (III), under amide forming conditions.

In a further aspect, process B may include as a preliminary step (a) reacting a compound of formula (IV) with a compound of formula (V), to form the intermediate ester (VI), which need not be isolated prior to treatment with the de-esterifying agent in step (a).

When Y is N, the pyrimidone of formula (IV) may be readily prepared by adapting a standard pyrimidone synthesis involving an amidine and a 1,3-dicarbonyl compound, by reacting an amidine of formula (VII): 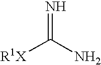
in which R¹ and X are as hereinbefore defined,
preferably as a salt thereof, for instance the hydrochloride salt,
with a compound of formula (VIII): 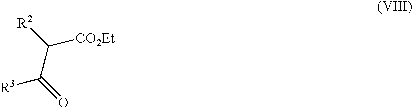
in which R² and R³ are as hereinbefore defined.

Alternatively, for pyrimidones in which X is (CH₂)ₙS, the intermediate compound of formula (IV) may be formed by reacting a compound of formula (VIII) with thiourea in the presence of sodium ethoxide (preferably generated in situ from sodium and ethanol), followed by alkylation with R¹L in which R¹ and L are as hereinbefore described. Conditions for the alkylation reaction typically include thioether forming conditions. Advantageously, the reaction is carried out in the presence of a base such as sodium ethoxide or potassium carbonate, preferably in a solvent such as ethanol or dimethylformamide, or a secondary or tertiary amine base such as di-isopropylethylamine, in a solvent such as dichloromethane.

When Y is CH, the overall synthesis of the compounds of formula (I) is illustrated in the following scheme:

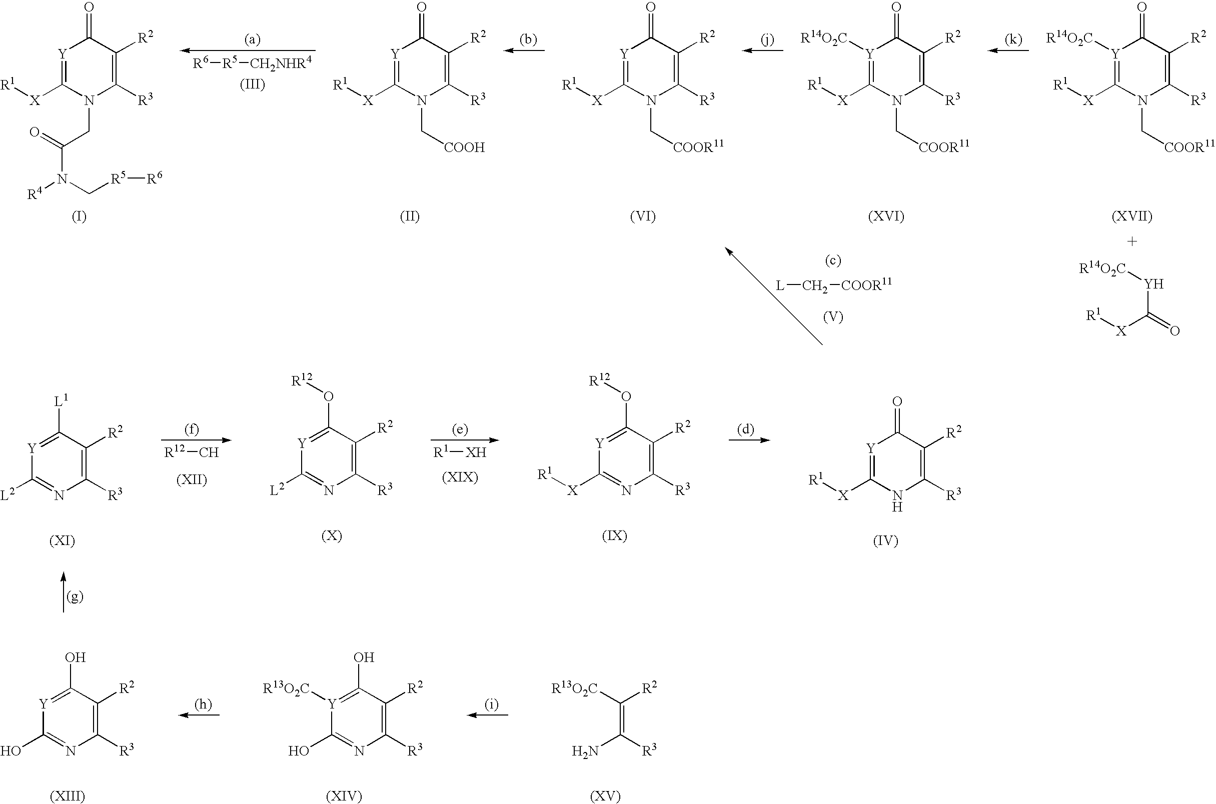

Referring to the scheme, the ester (VI) is usually prepared by N-1 alkylation of (IV) using (V) in which R¹¹ as hereinbefore defined, e.g. (V) is t-butyl bromoacetate or ethyl bromoacetate, in the presence of a base e.g. BuLi in THF or diisopropylethylamine in dichloromethane (step c).

When X is CH₂S, the R¹X substituent is preferably introduced by displacement of a leaving group L² (e.g. Cl) (step e) on a pyridine (X), to give the 2-substituted pyridine (IX). Transformation of (IX) to the 4-pyridone (IV) is accomplished by deprotection of the 4-oxygen (e.g. using Ph₃P)₃RhCl when in aq. ethanol when R¹²=allyl) (step d). The pyridine (X) may be prepared by steps (i), (h), (g) and (f), in which:
(f) treatment of (XP with R¹²OH (XI), in which R¹² is e.g. C₍₁₋₆₎alkyl or allyl, and sodium hydride in DMF;
(g) treatment of (XIII) with phosphorus oxychloride;
(h) treatment of (XIV) with aq HCl with heating;
(i) treatment of (XV) with di-lower alkyl malonate and sodium alkoxide in alcohol (in which R¹³ is C₍₁₋₆₎allyl, typically R¹³=Et); and
R¹—CH₂SH (XIX) is typically prepared from the thioacetate, which is formed from the corresponding alkyl bromide R₁—CH₂Br.

When X is alkylene, it is preferable to use steps (j) and (k) (intermediates (XVI), (XVII), (XVIII)) in which the 3-ester group is removed from intermediate (XVI) R¹⁴=C₍₁₋₆₎alkyl by heating in diphenyl ether where R¹⁴=tBu (step j). Intermediate (XVI) is formed from the 2,6-dioxo-1,3-oxazine (XVII) and ester (XVIII) by treatment with a base (NaH) in DMF.

It will be appreciated that compounds of formula (I) may also be prepared from other compounds of formula (I) using conventional interconversion procedures. Thus, a process for preparing a compound of formula (I) by interconversion of another compound of formula (I) (process C) constitutes a further aspect of the present invention.

It will be appreciated by those skilled in the art that it may be desirable to use protected derivatives of intermediates used in the preparation of compounds of formula (I). Thus, the above processes may require deprotection as an intermediate or final step to yield the desired compound. Thus, according to another process (D), a compound of formula (I) may be prepared by subjecting a protected derivative of a compound of formula (I) to reaction to remove the protecting group or groups present, constituting a further aspect of the present invention.

Protection and deprotection of functional groups may be effected using conventional means. Thus, hydroxyl groups may be protected using any conventional hydroxyl protecting group, for example, as described in Protective Groups in Organic Chemistry, Ed. J. F. W. McOmie (Plenum Press, 1973) or Protective Groups in Organic Synthesis by Theodora W. Green (John Wiley and Sons, 1991).

Examples of suitable hydroxyl protecting groups includes groups selected from alkyl (e.g. t-butyl or methoxymethyl), aralkyl (e.g. benzyl, diphenylmethyl or triphenylmethyl), heterocyclic groups such as tetrahydropyranyl, acyl (e.g. acetyl or benzoyl) and silyl groups such as trialkylsilyl (e.g. t-butyldimethylsilyl). The hydroxyl protecting groups may be removed by conventional techniques. Thus, for example alkyl, silyl, acyl and heterocyclic groups may be removed by solvolysis, e.g. by hydrolysis under acidic or basic conditions. Aralkyl groups such as triphenylmethyl may be similarly be removed by solvolysis, e.g. by hydrolysis under acidic conditions. Aralkyl groups such as benzyl may be cleaved by hydrogenolysis in the presence of a Noble metal catalyst such as palladium-on-charcoal.

The present invention will now be illustrated by the following example.

### EXAMPLE 1

### N-(2-Diethylaminoethyl)-2-(2-(2,3-difluorophenyl)ethyl)-4-oxo-5,7-dihydro-4H-thieno[3,4-d]pyrimidin-1-yl)-N-4-(4-trifluoromethyl-phenyl)benzyl)acetamide bitartrate

2-(2-(2,3-Difluorophenyl)ethyl)-5,7-dihydro-1H-thieno[3,4-d]pyrimidin-4-one

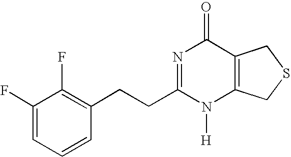

To a solution of 3-(2,3-difluorophenyl)-propionamidine hydrochloride (0.627 g, 2.84 mmol) (prepared according to the general procedure of Andrews et al., Mol. Cryst. Liq. Cryst., 1985, 123, 257-270) in ethanol (10 ml) was added sodium hydride (0.12 g, 3 mmol, 60% in paraffin) portionwise. After stirring for 15 min, methyl-4-oxo-tetrahydrothienyl-3-carboxylate (0.45 g, 2.8 mmol) (Maybridge Chemical Co. Ltd.) was added and the solution heated at reflux for 18 h. The solution was allowed to cool then concentrated and the residues chromatographed over silica eluting with dichloromethane followed by ethyl acetate to yield the title compound as a cream solid (0.379 g, 45%). ¹H-NMR (d₆-DMSO): δ 2.85 (2H, t), 3.05 (2H, t), 3.92 (2H, br), 4.08 (2H, br), 7.11 (2H, m), 7.3 (1H, m), 12.6 (1H, br s). MS (APCI+) found (M+1)=295; C₁₄H₁₂F₂N₂OS requires 294
Ethyl(2-(2-(2,3-difluorophenyl)ethyl)-4-oxo-5,7-dihydro-4H-thieno[3,4-d]pyrimidin-1-yl)acetate

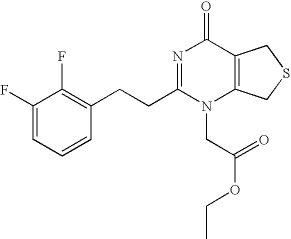

A solution of 2-(2-(2,3-difluorophenyl)ethyl)-5,7-dihydro-1H-thieno[3,4-d]pyrimidin-4-one (0.7 g, 2.38 mmol), ethyl bromoacetate (1.19 g, 7.14 mmol) and diisopropylethylamine (1.24 ml, 7.14 mmol) in dichloromethane (15 ml) was stirred for 5 days, washed with 2M hydrochloric acid solution then dried MgSO₄) and concentrated. Chromatography of the residues over silica eluting with a gradient from dichloromethane to dichloromethane/ether 4:1 yielded the title compound (0.10 g, 11%). ¹H-NMR (d₆-DMSO): δ 1.23 (3H, t), 2.35 (1H, t), 2.78 (1H, t), 2.9-3.1 (2H, m), 3.92 (1H, br), 4.14.3 (6H, br m), 5.07 (2H, s), 7.2 (3H, m). MS (APCI+) found (M+1)=381; C₁₈H₁₈F₂N₂O₃S requires 380.
(2-(2-(2,3-Difluorophenyl)ethyl)-4-oxo-5,7-dihydro-4H-thieno[3,4-d]pyrimidin-1-yl)acetic acid

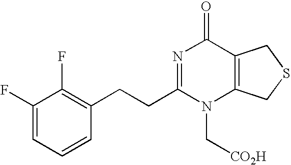

A solution of ethyl(2-(2-(2,3-difluorophenyl)ethyl)-4-oxo-5,7-dihydro-4H-thieno[3,4-d]pyrimidin-1-yl)acetate (0.1 g, 0.26 mmol) and sodium hydroxide (0.015 g, 0.37 mmol) in 1:1 dioxan/water (4 ml) was stirred for 3 h then acidified with 2M hydrochloric acid and concentrated. The residues were washed with water then dichloromethane and then taken up in acetone and dried (MgSO₄) and concentrated to yield the title compound (33 mg, 36%). ¹H-NMR (d₆-DMSO): δ 3.0(4H, m), 3.8 (2×, br), 4.2 (2H, br), 4.8 (2×, s), 7.0-7.3 (4H, m). MS (APCI−) found (M−1)=351; C₁₆H₁₄F₂N₂O₃S requires 352.

N-(2-Diethylaminoethyl)-2-(2-(2,3-difluorophenyl)ethyl)-4-oxo-5,7-dihydro-4H-thieno[3,4-d]pyrimidin-1-yl)-N-4-(4-trifluoromethylphenyl)benzyl)acetamide bitartrate

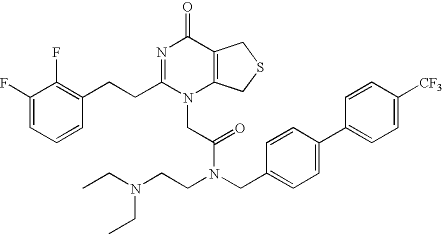

A solution of (2-(2-(2,3-difluorophenyl)ethyl)-4-oxo-5,7-dihydro-4H-thieno[3,4-d]pyrimidin-1-yl)acetic acid (0.03 g, 0.09 mmol), N-(2-diethylaminoethyl)-4-(4-trifluoromethylphenyl)benzylamine (WO 00/66567) (0.03 g, 0.09 mmol) and 1-(3-dimethylaminopropyl)-3-ethylcarbodiimide (DEC) (0.032 g, 0.17 mmol) in dimethylformamide (2 ml) was stirred for 18 h then concentrated. The residues were separated between ethyl acetate and sodium bicarbonate solution and the organics isolated, dried (MgSO₄) and concentrated to yield the amide. The bitartrate salt was formed by dissolving the amide with tartaric acid (0.012 g, 0.09 mmol) in methanol and concentrating the solution to provide the title compound as the bitartrate salt (0.067 g, 100%).

Free Base Data:

¹H-NMR (d₆-DMSO): δ 0.96 (6H, t), 2.2-2.7 (10H, m), 2.8-4.3 (811, m), 5.2 (2H, m), 7.1 (2H, m), 7.25 (1H, m), 7.5 (2H, d), 7.65 (2H, d), 7.8 (2H, d), 7.9 (2H, d). MS (APCI−) found (M−1)=683; C₃₆H₃₇F₅N₄O₂S requires 684.

Bitartrate Data:

¹H-NMR (d₄-MeOH): (selected peaks) δ 1.0-1.4 (6H, m), 4.37 (2H, m), 6.9-7.2 (3H, m), 7.35-7.9 (8H, m).

Biological Data

1. Screen for Lp-PLA₂ Inhibition.

Enzyme activity was determined by measuring the rate of turnover of the artificial substrate (A) at 37° C. in 50 mM HEPES (N-2-hydroxyethylpiperazine-N′-2-ethanesulphonic acid) buffer containing 150 mM NaCl, pH 7.4.

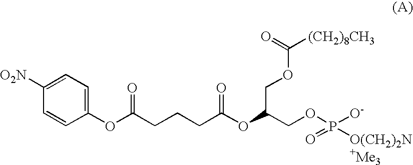

Assays were performed in 96 well titre plates.

Recombinant Lp-PLA₂ was purified to homogeneity from baculovirus infected Sf9 cells, using a zinc chelating column, blue sepharose affinity chromatography and an anion exchange column. Following purification and ultrafiltration, the enzyme was stored at 6 mg/ml at 4° C. Assay plates of compound or vehicle plus buffer were set up using automated robotics to a volume of 170 μl. The reaction was initiated by the addition of 20 μl of 10× substrate (A) to give a final substrate concentration of 20 μM and 10 pd of diluted enzyme to an approximate final 0.1 nM Lp-PLA₂. The reaction was followed at 405 nm and 37° C. for 20 minutes using a plate reader with automatic mixing. The rate of reaction was measured as the rate of change of absorbance.

Results

The compounds described in the Examples were tested as described above and had IC₅₀ values in the range <0.1 to 100 nM.

1. A compound of formula (I): 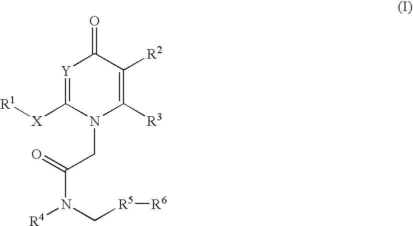
in which:
R¹ is an aryl group, optionally substituted by 1, 2, 3 or 4 substituents which may be the same or different selected from the group consisting of C₍₁₋₆₎alkyl, C₍₁₋₆₎alkoxy, C₍₁₋₆₎alkylthio, hydroxy, halogen, CN, and mono to perfluoro-C₍₁₋₄₎alkyl;
R² and R³ together with the ring carbon atoms to which they are attached form a fused 5- or 6-membered non-aromatic heterocyclyl ring containing 1 or 2 heteroatoms which may be the same or different selected from N, O and S, optionally substituted by 1, 2 or 3 substituents which may be the same or different selected from the group consisting of oxo, hydroxy, halogen, OR⁷, COR⁷, COOR⁷, CONR⁹R¹⁰, SR⁷, NR⁷COR⁸, SO₂NR⁹R¹⁰, NR⁷SO₂R⁸, and C₍₁₋₆₎alkyl optionally substituted by 1, 2 or 3 substituents selected from the group consisting of hydroxy, halogen, OR⁷, COR⁷, carboxy, COOR⁷, CONR⁹R¹⁰ and NR⁹R¹⁰;
R⁴ is hydrogen, C₍₁₋₆₎alkyl which may be unsubstituted or substituted by 1, 2 or 3 substituents selected from the group consisting of hydroxy, halogen, OR⁷, COR⁷, carboxy, COOR⁷, CONR⁹R¹⁰, NR⁹R¹⁰, NR⁷COR⁸, mono- or di-(hydroxyC₍₁₋₆₎alkyl)amino and N-hydroxyC₍₁₋₆₎alkyl-N—C₍₁₋₆₎alkylamino; or
R⁴ is Het-C₍₀₋₄₎alkyl in which Het is a 5- to 7-membered heterocyclyl ring comprising N and optionally O or S, and in which N may be substituted by COR⁷, COOR⁷, CONR⁹R¹⁰, or C₍₁₋₆₎alkyl optionally substituted by 1, 2 or 3 substituents selected from the group consisting of hydroxy, halogen, OR⁷, COR⁷, carboxy, COOR⁷, CONR⁹R¹⁰ and NR⁹R¹⁰;
R⁵ is an aryl or a heteroaryl ring optionally substituted by 1, 2, 3 or 4 substituents which may be the same or different selected from the group consisting of C₍₁₋₆₎alkyl, C₍₁₋₆₎alkoxy, C₍₁₋₆₎alkylthio, arylC₍₁₋₆₎alkoxy, hydroxy, halogen, CN, COR⁷, carboxy, COOR⁷, NR⁷COR⁸, CONR⁹R¹⁰, SO₂NR⁹R¹⁰, NR⁷SO₂R⁸, NR⁹R¹⁰, mono to perfluoro-C₍₁₋₄₎alkyl and mono to perfluoro-C₍₁₋₄₎alkoxy;
R⁶ is an aryl or a heteroaryl ring which is further optionally substituted by 1, 2, 3 or 4 substituents which may be the same or different selected from the group consisting of C₍₁₋₁₈₎alkyl, C₍₁₋₁₈₎alkoxy, C₍₁₋₆₎alkylthio, C₍₁₋₆₎alkylsulfonyl, arylC₍₁₋₆₎alkoxy, hydroxy, halogen, CN, COR⁷, carboxy, COOR⁷, CONR⁹R¹⁰, NR⁷COR⁸, SO₂NR⁹R¹⁰, NR⁷SO₂R⁸, NR⁹R¹⁰, mono to perfluoro-C₍₁₋₄₎alkyl and mono to perfluoro-C₍₁₋₄₎alkoxy, or C₍₅₋₁₀₎alkyl;
R⁷ and R⁸ are independently hydrogen or C₍₁₋₁₂₎alkyl;
R⁹ and R¹⁰ which may be the same or different is each selected from the group consisting of hydrogen, or C₍₁₋₁₂₎alkyl, or R⁹ and R¹⁰ together with the nitrogen to which they are attached form a 5- to 7 membered ring optionally containing one or more further heteroatoms selected from the group consisting of oxygen, nitrogen and sulphur, and optionally substituted by one or two substituents selected from hydroxy, oxo, C₍₁₋₄₎alkyl, C₍₁₋₄₎alkylcarboxy, aryl, e.g. phenyl, or aralkyl;
X is C₍₂₋₄₎alkylene, optionally substituted by 1, 2 or 3 substituents selected from the group consisting of methyl and ethyl, CH═CH or (CH₂)ₙS where n is 1, 2 or 3; and
Y is CH or N; and
pharmaceutically acceptable salts thereof.

2. A compound according to claim 1 wherein R¹ is phenyl optionally substituted by halogen, C₍₁₋₆₎alkyl, trifluoromethyl or C₍₁₋₆₎alkoxy.

3. A compound according to claim 1 wherein R² and R³ together with the ring carbon atoms to which they are attached form a fused non-aromatic 5- or 6-membered heterocyclyl ring containing a sulphur atom, a nitrogen atom or an oxygen atom.

4. A compound according to claim 1 wherein R⁴ is hydrogen, methyl, 2-(diethylamino)ethyl, 2-(piperidin-1-yl)ethyl, 2-(pyrrolidin-1-yl)ethyl, 1-methyl-piperidinyl, 1-ethyl-piperidin-4-yl, 1-ethyl-pyrrolidin-2-ylmethyl or 1-(2-methoxyethyl)piperidin-4-yl.

5. A compound according to claim 1 wherein R⁵ is phenyl.

6. A compound according to claim 1 wherein R⁶ is phenyl optionally substituted by halogen or trifluoromethyl.

7. A compound according to claim 1 wherein X is C₍₂₋₄₎alkylene.

8. A compound according to claim 1 wherein Y is N.

9. A compound according to claim 1 which is N-(2-Diethylaminoethyl)-2-(2-(2,3-difluorophenyl)ethyl)-4-oxo-5,7-dihydro-4H-thieno[3,4-d]pyrimidin-1-yl)-N-4-(4-trifluoromethylphenyl)benzyl)acetamide.

10. A pharmaceutical composition comprising a compound of formula (I) as claimed in claim 1 and a pharmaceutically acceptable carrier.

11. (Deleted)

12. (Deleted)

13. A method of treating a disease state associated with activity of the enzyme Lp-PLA₂ which method involves treating a patient in need thereof with a therapeutically effective amount of a compound of formula (I) as claimed in claim 1.

14. A process for preparing a compound of formula (I) as defined in claim 1 which process comprises reacting an acid compound of formula (II): 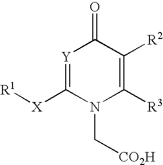
in which X, Y, R¹, R² and R³ are as hereinbefore defined,
with an amine compound of formula (III):
R⁶—R⁵—CH₂NHR⁴ (III)
in which R⁴, R⁵ and R⁶ are as hereinbefore defined; under amide forming conditions.
